(19) United States
(12) Patent Application Publication
(10) Pub. No.: US 2015/0335662 A1
(43) Pub. Date: Nov. 26, 2015

(54) METHODS FOR DIAGNOSING AND TREATING SEPSIS

(71) Applicant: LOMA LINDA UNIVERSITY, Loma Lima, CA (US)

(72) Inventors: Hung Bryant Nguyen, Redlands, CA (US); David J. Baylink, Redlands, CA (US); Kin-Hing William Lau, Redlands, CA (US)

(21) Appl. No.: 14/761,939

(22) PCT Filed: Jan. 17, 2014
(86) PCT No.: PCT/US14/12130

§ 371 Date: Jul. 17, 2015

Related U.S. Application Data
(63) Continuation-in-part of application No. PCT/US2013/035025, filed on Apr. 2, 2013.
Application No. PCT/US2013/035025 is a continuation-in-part of application No. PCT/US2013/053186, filed on Aug. 1, 2013.
(60) Provisional application No. 61/754,382, filed on Jan. 18, 2013.
(60) Provisional application No. 61/755,383, filed on Jan. 22, 2013.
(60) Provisional application No. 61/759,277, filed on Jan. 31, 2013.
(60) Provisional application No. 61/767,095, filed on Feb. 20, 2013.
(60) Provisional application No. 61/816,627, filed on Apr. 26, 2013.

(51) Int. Cl. A61K 31/593; G01N 33/82; A61K 31/592

(52) U.S. Cl. CPC A61K 31/593; A61K 31/592; G01N 33/82; G01N 2800/26; G01N 2800/56

(57) ABSTRACT

A method for treating a patient with an episode of sepsis. A method for categorizing the severity of an episode of sepsis in a patient.

### CROSS-REFERENCE TO RELATED APPLICATIONS

The present Application claims priority from International Patent Application No. PCT/US2013/053186 titled “Compositions and Methods for Diagnosing and Treating Sepsis,” filed Aug. 1, 2013, which claims priority from International Patent Application No. PCT/US2013/035025 titled “Compositions and Methods for Diagnosing and Treating Sepsis,” filed Apr. 2, 2013, which claims the benefit of U.S. Provisional Patent Application 61/754,382, titled “Methods and Substances for Diagnosing and Treating Sepsis,” filed Jan. 18, 2013; U.S. Provisional Patent Application 61/755,383, titled “Substances and Methods for Diagnosing and Treating Sepsis,” filed Jan. 22, 2013; U.S. Provisional Patent Application 61/759,277, titled “Compositions and Methods for Diagnosing and Treating Sepsis,” filed Jan. 31, 2013; U.S. Provisional Patent Application 61/767,095, titled “Compositions and Methods for Diagnosing and Treating Sepsis,” filed Feb. 20, 2013; and U.S. Provisional Patent Application 61/816,627, titled “Compositions and Methods for Diagnosing and Treating Sepsis,” filed Apr. 26, 2013; the contents of which are incorporated in this disclosure by reference in their entirety.

### BACKGROUND

Sepsis is a whole-body inflammatory state produced in response to an infective agent, such as for example bacteria, fungi, parasites or viruses. Sepsis commonly presents with inflammatory signs and symptoms related to an immune response to the infective agent, and typically include one or more than one sign and symptom selected from the group consisting of altered mentation, edema, flushing, hyperthermia, hypothermia, hypotension, hyperventilation, lightheadedness and tachycardia. More than 750,000 people in the United States are admitted annually to intensive care units (ICU) with the diagnosis of sepsis. Between one third and one half of sepsis patients progress to multiple organ dysfunction syndrome (MODS), (“multiple organ failure” (MOF), and “multisystem organ failure” (MSOF)) leading to an overall mortality of between 25% and 30%, or about 200,000 deaths in the United States annually, making sepsis the tenth leading cause of mortality in the United States. Estimated costs for treating sepsis exceed twelve billion dollars annually in the United States.

The treatment of an episode of sepsis usually involves antibiotics, antifungal agents, antiparasitic agents or antiviral agents where the underlying infective agent is determined, and cardiovascular support with intravenous fluids and vasopressors to maintain blood pressure. In episodes of sepsis that progress to multiple organ dysfunction syndrome, mechanical ventilation and dialysis can be used to support the function of the lungs and kidneys, respectively. Despite these treatment modalities, sepsis continues to result in high mortality.

Additionally, there is no reliable test for clinicians to determine which patients will progress from an episode of sepsis without multiple organ dysfunction syndrome to an episode of sepsis with multiple organ dysfunction syndrome. Having a reliable test for predicting progress from an episode of sepsis without multiple organ dysfunction syndrome to an episode of sepsis with multiple organ dysfunction syndrome would allow physicians to test new treatment modalities, and allow physicians to intervene more aggressively where indicated.

Therefore, there is a need for a new method for determining which patients will progress from sepsis without multiple organ dysfunction syndrome to sepsis with multiple organ dysfunction syndrome. Further, there is a need for a new method for treating a patient with an episode of sepsis.

### SUMMARY

According to one embodiment of the present invention, there is provided a method for treating a patient with an episode of sepsis, where treating comprises either decreasing the severity of an episode of sepsis in the patient, or decreasing the likelihood that the patient with sepsis will progress to sepsis with multiple organ dysfunction syndrome, or decreasing the likelihood that the patient with an episode of sepsis will die from the episode of sepsis, or a combination of the preceding. The method comprises: a) determining that the patient has an episode of sepsis; b) determining a blood calcium level in the patient; c) confirming that the patient has hypocalcemia or normocalcemia but not hypercalcemia; and d) administering to the patient one or more than one dose of an agent or a composition, where the agent is a chemical that raises the blood calcium level in the patient, or the composition comprises an agent that is a chemical that raises the blood calcium level in the patient, thereby treating the patient. In one embodiment, the method further comprises e) determining one or more than one subsequent blood calcium level in the patient; and f) additionally administering to the patient one or more than one dose of the agent or the composition if the one or more than one subsequent blood calcium level in the patient is hypocalcemia or normocalcemia but ceasing the administration of the agent or the composition if the one or more than one subsequent blood calcium level in the patient is hypercalcemia In one embodiment, hypocalcemia is defined as less than 9.0 mg/dL or less than 2.2 mmol/L, normocalcemia is defined as between 9.0 and 10.5 mg/dL or between 2.2 and 2.6 mmol/L, and hypercalcemia is defined as greater than 10.5 mg/dL or greater than 2.6 mmol/L.

According to another embodiment of the present invention, there is provided another method for treating a patient with an episode of sepsis, where treating comprises either decreasing the severity of an episode of sepsis in the patient, or decreasing the likelihood that the patient with sepsis will progress to sepsis with multiple organ dysfunction syndrome, or decreasing the likelihood that the patient with an episode of sepsis will die from the episode of sepsis, or a combination of the preceding. The method comprises: a) determining that the patient has an episode of sepsis; b) determining a first blood calcium level in the patient; c) confirming that the patient has hypocalcemia or normocalcemia but not hypercalcemia; and d) administering to the patient one or more than one dose of an agent or a composition, where the agent is a chemical that raises the blood calcidiol level in the patient, or the composition comprises an agent that is a chemical that raises the blood calcidiol level in the patient, thereby treating the patient; e) determining one or more than one subsequent blood calcium level in the patient; and f) additionally administering to the patient one or more than one dose of the agent or the composition if the one or more than one subsequent blood calcium level in the patient is hypocalcemia or normocalcemia but ceasing the administration of the agent or the composition if the one or more than one subsequent blood calcium level in the patient is hypercalcemia. In one embodiment, hypocalcemia is defined as less than 9.0 mg/dL or less than 2.2 mmol/L, normocalcemia is defined as between 9.0 and 10.5 mg/dL or between 2.2 and 2.6 mmol/L, and hypercalcemia is defined as greater than 10.5 mg/dL or greater than 2.6 mmol/L.

According to another embodiment of the present invention, there is provided another method for treating a patient with an episode of sepsis, where treating comprises either decreasing the severity of an episode of sepsis in the patient, or decreasing the likelihood that the patient with sepsis will progress to sepsis with multiple organ dysfunction syndrome, or decreasing the likelihood that the patient with an episode of sepsis will die from the episode of sepsis, or a combination of the preceding. The method comprises: a) determining that the patient has an episode of sepsis; b) determining a first blood calcium level in the patient; c) confirming that the patient has hypocalcemia or normocalcemia but not hypercalcemia; d) administering to the patient one or more than one dose of an agent or a composition, where the agent is a chemical that raises the blood calcitriol level in the patient, or the composition comprises an agent that is a chemical that raises the blood calcitriol level in the patient, thereby treating the patient; e) determining one or more than one subsequent blood calcium level in the patient; and f) additionally administering to the patient one or more than one dose of the agent or the composition if the one or more than one subsequent blood calcium level in the patient is hypocalcemia or normocalcemia but ceasing the administration of the agent or the composition if the one or more than one subsequent blood calcium level in the patient is hypercalcemia. In one embodiment, hypocalcemia is defined as less than 9.0 mg/dL or less than 2.2 mmol/L, normocalcemia is defined as between 9.0 and 10.5 mg/dL or between 2.2 and 2.6 mmol/L, and hypercalcemia is defined as greater than 10.5 mg/dL or greater than 2.6 mmol/L.

According to another embodiment of the present invention, there is provided another method for treating a patient with an episode of sepsis, where treating comprises either decreasing the severity of an episode of sepsis in the patient, or decreasing the likelihood that the patient with sepsis will progress to sepsis with multiple organ dysfunction syndrome, or decreasing the likelihood that the patient with an episode of sepsis will die from the episode of sepsis, or a combination of the preceding. The method comprises: a) determining that the patient has an episode of sepsis; b) determining a blood calcidiol level in the patient; c) confirming that the patient has a sub-normal blood calcidiol level or a normal blood calcidiol level but not an elevated blood calcidiol level; d) administering to the patient one or more than one dose of an agent or a composition, where the agent is a chemical that raises the blood calcidiol level in the patient, or the composition comprises an agent that is a chemical that raises the blood calcidiol level in the patient, thereby treating the patient; e) determining one or more than one subsequent blood calcidiol level in the patient; and f) additionally administering to the patient one or more than one dose of the agent or the composition if the one or more than one subsequent blood calcidiol level in the patient is sub-normal blood calcidiol level or normal blood calcidiol level but ceasing the administration of the agent or the composition if the one or more than one subsequent blood calcidiol level in the patient is elevated blood calcidiol level. In one embodiment, the sub-normal blood calcidiol level is defined as less than 30 ng/ml, the normal blood calcidiol level is defined as between 30 and 80 ng/ml, and the elevated blood calcidiol level is defined as greater than 80 ng/ml.

According to another embodiment of the present invention, there is provided another method for treating a patient with an episode of sepsis, where treating comprises either decreasing the severity of an episode of sepsis in the patient, or decreasing the likelihood that the patient with sepsis will progress to sepsis with multiple organ dysfunction syndrome, or decreasing the likelihood that the patient with an episode of sepsis will die from the episode of sepsis, or a combination of the preceding. The method comprises: a) determining that the patient has an episode of sepsis; b) determining a blood calcitriol level in the patient; c) confirming that the patient has a sub-normal blood calcitriol level or a normal blood calcitriol level but not an elevated blood calcitriol level; and d) additionally administering to the patient one or more than one dose of the agent or the composition if the one or more than one subsequent blood calcitriol level in the patient is sub-normal blood calcidiol level or normal blood calcidiol level but ceasing the administration of the agent or the composition if the one or more than one subsequent blood calcitriol level in the patient is elevated blood calcidiol level. In one embodiment, the sub-normal blood calcitriol level is defined as less than 20 μg/ml, the normal blood calcitriol level is defined as between 20 and 50 pg/ml, and the elevated blood calcitriol level is defined as greater than 50 ng/ml.

In one embodiment, the one or more than one dose administered is between 0.01 ng and 1 gram. In another embodiment, the one or more than one dose administered is between 0.01 ng and 10,000 mcg. In another embodiment, the one or more than one dose administered is between 1 ng and 10,000 mcg. In another embodiment, the one or more than one dose administered is between 0.1 mcg and 10,000 mcg. In another embodiment, the one or more than one dose administered is between 1 mcg and 1,000 mcg. In another embodiment, the one or more than one dose administered is between 100 units to 10,000,000 units. In another embodiment, the one or more than one dose administered is between 400 units to 10,000,000 units. In another embodiment, the one or more than one dose administered is between 1000 units to 10,000,000 units. In another embodiment, the one or more than one dose administered is between 1000 units to 1,000,000 units. In another embodiment, the one or more than one dose administered is 1000 units to 100,000 units. In another embodiment, the one or more than one dose administered is between 1000 units to 10,000 units. In another embodiment, the one or more than one dose is administered by a route selected from the group consisting of intrarectal, intramuscular, intraperitoneal, intrathecal, intravenous and oral. In another embodiment, the one or more than one dose is a plurality of doses. In another embodiment, the plurality of doses is two doses. In another embodiment, the plurality of doses is three doses. In another embodiment, the plurality of doses is four doses. In another embodiment, the plurality of doses is more than four doses. In another embodiment, the plurality of doses is twenty-eight doses. In another embodiment, the plurality of doses are administered between one hour and seventy-two hours apart. In another embodiment, the plurality of doses are administered between one hour and thirty-six hours apart. In another embodiment, the plurality of doses are administered between one hour and twenty-four hours apart. In another embodiment, the plurality of doses are administered twenty-four hours apart. In another embodiment, the plurality of doses are 2 mcg of paricalcitol administered once daily for twenty-eight days. In another embodiment, the agent is selected from the group consisting of 1 alpha-hydroxyergocalciferol; 1-alpha-hydroxyvitamin D2; 1-alpha-hydroxyvitamin D3; 1-alpha(OH)D2; 1-alpha(OH)D3; 1,24,25-trihydroxyvitamin D3; 1,25(OH)2-16-ene-23yne-D3; 19-nor-1,25-dihydroxyvitamin D2; 19-nor-1,25-(OH)2-vitamin D2; 19-nor-1,25-dihydroxyvitamin D3; 20(17→18)-abeo-1α,25-dihydroxy-22-homo-21-norvitamin D(3); 24,25-dihydroxyvitamin D3; alfacalcidiol; calcidiol; calcium; calcitriol; cholecalciferol; doxercalciferol; ergocalciferol; EB1089; HM; KH1060; MC1288 and parathyroid hormone. In another embodiment, the agent is a functional analog of one or more than one of the group of 1 alpha-hydroxyergocalciferol; 1-alpha-hydroxyvitamin D2; 1-alpha-hydroxyvitamin D3; 1-alpha(OH)D2; 1-alpha(OH)D3; 1,24,25-trihydroxyvitamin D3; 1,25(OH)2-16-ene-23yne-D3; 19-nor-1,25-dihydroxyvitamin D2; 19-nor-1,25-(OH)2-vitamin D2; 19-nor-1,25-dihydroxyvitamin D3; 20(17→18)-abeo-1α,25-dihydroxy-22-homo-21-norvitamin D(3); 24,25-dihydroxyvitamin D3; alfacalcidiol; calcidiol; calcium; calcitriol; cholecalciferol; doxercalciferol; ergocalciferol; EB1089; HM; KH1060; MC1288 and parathyroid hormone. In another embodiment, the agent is calcitriol.

In another embodiment, the composition comprises at least two agents. In another embodiment, the composition comprises at least two agents selected from the group consisting of 1 alpha-hydroxyergocalciferol; 1-alpha-hydroxyvitamin D2; 1-alpha-hydroxy vitamin D3; 1-alpha(OH)D2; 1-alpha(OH)D3; 1,24,25-trihydroxyvitamin D3; 1,25(OH)2-16-ene-23yne-D3; 19-nor-1,25-dihydroxyvitamin D2; 19-nor-1,25-(OH)2-vitamin D2; 19-nor-1,25-dihydroxyvitamin D3; 20(17→18)-abeo-1α,25-dihydroxy-22-homo-21-norvitamin D(3); 24,25-dihydroxyvitamin D3; alfacalcidiol; calcidiol; calcium; calcitriol; cholecalciferol; doxercalciferol; ergocalciferol; EB1089; HM; KH1060; MC1288, parathyroid hormone, and a functional equivalent of 1-alpha-hydroxyvitamin D2; 1-alpha-hydroxyvitamin D3; 1-alpha(OH)D2; 1-alpha(OH)D3; 1,24,25-trihydroxyvitamin D3; 1,25(OH)2-16-ene-23yne-D3; 19-nor-1,25-dihydroxyvitamin D2; 19-nor-1,25-(OH)2-vitamin D2; 19-nor-1,25-dihydroxyvitamin D3; 20(17→18)-abeo-1α,25-dihydroxy-22-homo-21-norvitamin D(3); 24,25-dihydroxyvitamin D3; alfacalcidiol; calcidiol; calcium; calcitriol; cholecalciferol; doxercalciferol; ergocalciferol; EB1089; HM; KH1060; MC1288, and parathyroid hormone, and a functional analog of the preceding.

According to another embodiment of the present invention, there is provided a method for characterizing the severity of an episode of sepsis in a patient, by determining a likelihood that a patient with an episode of sepsis will progress to sepsis with multiple organ dysfunction syndrome, or by determining a likelihood that a patient with an episode of sepsis will die from the episode of sepsis. The method comprises: a) determining that the patient has an episode of sepsis; and b) determining a blood level of calcium in the patient; where a blood level of calcium below a predetermined amount indicates an increased likelihood that a patient with an episode of sepsis will progress to sepsis with multiple organ dysfunction syndrome, or that the patient will die from the episode of sepsis. In one embodiment, the predetermined blood level of calcium is less than 9.0 mg/dL or less than 2.2 mmol/L.

According to another embodiment of the present invention, there is provided another method for characterizing the severity of an episode of sepsis in a patient, by determining a likelihood that a patient with an episode of sepsis will progress to sepsis with multiple organ dysfunction syndrome, or by determining a likelihood that a patient with an episode of sepsis will die from the episode of sepsis. The method comprises: a) determining that the patient has an episode of sepsis; and b) determining a blood level of calcidiol in the patient; where a blood level of calcidiol below a predetermined amount indicates an increased likelihood that a patient with an episode of sepsis will progress to sepsis with multiple organ dysfunction syndrome, or that the patient will die from the episode of sepsis. In one embodiment, the predetermined blood level of calcidiol is less than 30 ng/ml.

According to another embodiment of the present invention, there is provided another method for characterizing the severity of an episode of sepsis in a patient, by determining a likelihood that a patient with an episode of sepsis will progress to sepsis with multiple organ dysfunction syndrome, or by determining a likelihood that a patient with an episode of sepsis will die from the episode of sepsis. The method comprises: a) determining that the patient has an episode of sepsis; and b) determining a blood level of calcitriol in the patient; where a blood level of calcitriol below a predetermined amount indicates an increased likelihood that a patient with an episode of sepsis will progress to sepsis with multiple organ dysfunction syndrome, or that the patient will die from the episode of sepsis. In one embodiment, the predetermined blood level of calcitriol is less 30 pg/mL. In another embodiment, the predetermined blood level of calcitriol is less than 25 pg/mL. In another embodiment, the predetermined blood level of calcitriol is less than 20 pg/mL. In another embodiment, the predetermined blood level of calcitriol is less than 15 pg/mL. In another embodiment, the predetermined blood level of calcitriol is less than 14 pg/mL. In another embodiment, the predetermined blood level of calcitriol is less than 13.6 pg/mL. In another embodiment, the predetermined blood level of calcitriol is less than 10 pg/mL. In another embodiment, the predetermined blood level of calcitriol is less than 5 pg/mL.

In one embodiment, determining the patient has an episode of sepsis comprises referring to medical records relevant to the patient. In another embodiment, determining the patient has an episode of sepsis comprises diagnosing the episode of sepsis in the patient. In one embodiment, diagnosing the episode of sepsis in the patient comprises determining that the patient has the following criteria: 1) either i) a suspected or confirmed source of infection determined by the treating clinician, or ii) a serum lactate level greater than 2.5 mmol/L or both; and 2) two or more criteria selected from the group consisting of: a) a body temperature greater than 38° C. or less than 36° C.; b) i) a respiratory rate greater than 20 breaths per minute, or ii) partial pressure of carbon dioxide less than 32 mm Hg; c) a heart rate greater than 90 beats per minute; and d) i) a white blood cell count greater than 12,000 cells per mm³, or ii) a white blood cell count less than 4,000 cells per mm³, or iii) a white blood cell count comprising 10% or more than 10% immature forms. In one embodiment, determining that the patient has the following criteria comprises measuring the blood level in the patient a plurality of times. In one embodiment, the plurality of times is two times. In one embodiment, the plurality of times is three times. In one embodiment, the plurality of times is four times. In one embodiment, the plurality of times is more than four times. In one embodiment, the blood level in the patient is measured upon admission to a health care facility. In another embodiment, the blood level is measured upon diagnosing the patient with the episode of sepsis. In one embodiment, the calcitriol blood level is measured at one or more than one time selected from the group consisting of 0, 24, 48, 72 and 96 hours after admission to a health care facility. In another embodiment, the blood level is measured at one or more than one time selected from the group consisting of 0, 24, 48, 72 and 96 hours after diagnosing the patient with the episode of sepsis. In one embodiment, the method further comprises determining a presence of one or more than one patient criterion in the patient selected from the group consisting of age, Acute Physiology and Chronic Health Evaluation (APACHE) II scores, and a presence of stroke comorbidity, and where the presence of one or more than one criterion of an age greater than 70 years, an Acute Physiology and Chronic Health Evaluation II score greater than 25, and stroke comorbidity further increases the likelihood that a patient with an episode of sepsis will progress to sepsis with multiple organ dysfunction syndrome, or that the patient will die from the episode of sepsis.

### DRAWINGS

These and other features, aspects and advantages of the present invention will become better understood with regard to the following description, appended claims, and accompanying drawings where:

FIG. 1 is a table of baseline patient characteristics identified in patients studied according to the present invention relating the baseline patient characteristics to patient survival or patient nonsurvival (mortality);

FIG. 2 is a table of vitamin D status characteristics, and of other potentially relevant characteristics (creatinine, total calcium, albumin, and total bilirubin blood levels) measured at 0, 24, 48, and 72 hours from admission, and analyzed to determine their predictive value for patient survival and patient nonsurvival at thirty days from admission for sepsis;

FIG. 3 is a table of the predictive value of variables found to have potential relevance to patient survival and patient nonsurvival at thirty days from admission with sepsis;

FIG. 4 is a graph of receiver operating characteristics (ROC) curves that were generated to determine the predictive value of age, total calcium and calcitriol blood levels at 48 hours after admission with respect to patient survival and patient nonsurvival at thirty days from admission with sepsis;

FIG. 5 is a graph of Kaplan-Meir analysis performed to obtain patient survival curves over thirty days from admission for calcitriol blood levels, the variable with the highest area under the receiver operating characteristics curve;

FIG. 6 is a graph of the natural log of calcitriol blood levels as a function of the natural log of calcidiol blood levels for all patients;

FIG. 7 is a table of calcidiol blood levels and calcitriol blood levels in patient survivors and patient non-survivors from repeated measures regression analysis of the values in FIG. 6, showing that calcitriol production was lower in non-survivors for the same substrate calcidiol blood levels in survivors;

FIG. 8 is a graph of the natural log of calcitriol blood levels as a function of the natural log of parathyroid hormone (PTH) levels for all patients;

FIG. 9 is a graph of percent survival as a function of time after induction of sepsis in rats showing the effect of various doses of calcitriol (none, triangles; 25 ng daily, line; 100 ng daily, circles; and 400 ng daily, squares); and

FIG. 10 is a graph of body weight in grams as a function of time after induction of sepsis in rats showing the effect of various doses of calcitriol (none, triangles; 25 ng daily, line; 100 ng daily, circles; and 400 ng daily, squares).

### DESCRIPTION

According to one embodiment of the present invention, there is provided a method for treating a patient with an episode of sepsis, where treating comprises either decreasing the severity of an episode of sepsis in the patient, or decreasing the likelihood that the patient with sepsis will progress to sepsis with multiple organ dysfunction syndrome, or decreasing the likelihood that the patient with an episode of sepsis will die from the episode of sepsis, or a combination of the preceding. In one embodiment, the method comprises: a) determining that the patient has an episode of sepsis; b) determining a blood calcium level in the patient; c) confirming that the patient has hypocalcemia or normocalcemia but not hypercalcemia; and d) administering to the patient one or more than one dose of an agent or a composition, where the agent is a chemical that raises the blood calcium level in the patient, or the composition comprises an agent that is a chemical that raises the blood calcium level in the patient, thereby treating the patient. In another embodiment, the method further comprises e) determining one or more than one subsequent blood calcium level in the patient; and f) additionally administering to the patient one or more than one dose of the agent or the composition if the one or more than one subsequent blood calcium level in the patient is hypocalcemia or normocalcemia but ceasing the administration of the agent or the composition if the one or more than one subsequent blood calcium level in the patient is hypercalcemia. In another embodiment, the method comprises: a) determining that the patient has an episode of sepsis; b) determining a first blood calcium level in the patient; c) confirming that the patient has hypocalcemia or normocalcemia but not hypercalcemia; and d) administering to the patient one or more than one dose of an agent or a composition, where the agent is a chemical that raises the blood calcidiol level in the patient, or the composition comprises an agent that is a chemical that raises the blood calcidiol level in the patient, thereby treating the patient; e) determining one or more than one subsequent blood calcium level in the patient; and f) additionally administering to the patient one or more than one dose of the agent or the composition if the one or more than one subsequent blood calcium level in the patient is hypocalcemia or normocalcemia but ceasing the administration of the agent or the composition if the one or more than one subsequent blood calcium level in the patient is hypercalcemia. In another embodiment, the method comprises: a) determining that the patient has an episode of sepsis; b) determining a first blood calcium level in the patient; c) confirming that the patient has hypocalcemia or normocalcemia but not hypercalcemia; d) administering to the patient one or more than one dose of an agent or a composition, where the agent is a chemical that raises the blood calcitriol level in the patient, or the composition comprises an agent that is a chemical that raises the blood calcitriol level in the patient, thereby treating the patient; e) determining one or more than one subsequent blood calcium level in the patient; and f) additionally administering to the patient one or more than one dose of the agent or the composition if the one or more than one subsequent blood calcium level in the patient is hypocalcemia or normocalcemia but ceasing the administration of the agent or the composition if the one or more than one subsequent blood calcium level in the patient is hypercalcemia. In another embodiment, the method comprises: a) determining that the patient has an episode of sepsis; b) determining a blood calcidiol level in the patient; c) confirming that the patient has a sub-normal blood calcidiol level or a normal blood calcidiol level but not an elevated blood calcidiol level; d) administering to the patient one or more than one dose of an agent or a composition, where the agent is a chemical that raises the blood calcidiol level in the patient, or the composition comprises an agent that is a chemical that raises the blood calcidiol level in the patient, thereby treating the patient; e) determining one or more than one subsequent blood calcidiol level in the patient; and f) additionally administering to the patient one or more than one dose of the agent or the composition if the one or more than one subsequent blood calcidiol level in the patient is sub-normal blood calcidiol level or normal blood calcidiol level but ceasing the administration of the agent or the composition if the one or more than one subsequent blood calcidiol level in the patient is elevated blood calcidiol level. In another embodiment, the method comprises: a) determining that the patient has an episode of sepsis; b) determining a blood calcitriol level in the patient; c) confirming that the patient has a sub-normal blood calcitriol level or a normal blood calcitriol level but not an elevated blood calcitriol level; and d) additionally administering to the patient one or more than one dose of the agent or the composition if the one or more than one subsequent blood calcitriol level in the patient is sub-normal blood calcidiol level or normal blood calcidiol level but ceasing the administration of the agent or the composition if the one or more than one subsequent blood calcitriol level in the patient is elevated blood calcidiol level.

According to another embodiment of the present invention, there is provided a method for characterizing the severity of an episode of sepsis in a patient, by determining a likelihood that a patient with an episode of sepsis will progress to sepsis with multiple organ dysfunction syndrome, or by determining a likelihood that a patient with an episode of sepsis will die from the episode of sepsis. In one embodiment, the method comprises: a) determining that the patient has an episode of sepsis; and b) determining a blood level of calcium in the patient; where a blood level of calcium below a predetermined amount indicates an increased likelihood that a patient with an episode of sepsis will progress to sepsis with multiple organ dysfunction syndrome, or that the patient will die from the episode of sepsis. In another embodiment, the method comprises: a) determining that the patient has an episode of sepsis; and b) determining a blood level of calcidiol in the patient; where a blood level of calcidiol below a predetermined amount indicates an increased likelihood that a patient with an episode of sepsis will progress to sepsis with multiple organ dysfunction syndrome, or that the patient will die from the episode of sepsis. In another embodiment, the method comprises: a) determining that the patient has an episode of sepsis; and b) determining a blood level of calcitriol in the patient; where a blood level of calcitriol below a predetermined amount indicates an increased likelihood that a patient with an episode of sepsis will progress to sepsis with multiple organ dysfunction syndrome, or that the patient will die from the episode of sepsis. The methods will now be disclosed in detail.

As used in this disclosure, except where the context requires otherwise, the term “comprise” and variations of the term, such as “comprising,” “comprises” and “comprised”are not intended to exclude other additives, components, integers or steps.

As used in this disclosure, except where the context requires otherwise, the term “units” in reference to a dosage means “international units” (IU).

As used in this disclosure, except where the context requires otherwise, the method steps disclosed are not intended to be limiting nor are they intended to indicate that each step is essential to the method or that each step must occur in the order disclosed.

Except as indicated otherwise, all percents for components of the substances or components of the compositions according to the present invention are expressed in weight percents.

As used in this disclosure, “an agent according to the present invention” or “a composition according to the present invention” and similar phrases mean “one or more than one agent according to the present invention,” and “one or more than one composition according to the present invention,” respectively, except where the context requires otherwise.

As used in this disclosure, “sepsis” means a whole-body inflammatory state (“systemic inflammatory response syndrome” (SIRS)), produced in response to an infective agent, such as for example bacteria, fungi, parasites or viruses, where the whole-body inflammatory state presents with inflammatory signs and symptoms comprising one or more than one sign and symptom selected from the group consisting of altered mentation, bandemia, edema, flushing, hyperthermia, hypothermia, hypotension, hyperventilation, leukocytosis, leukopenia, lightheadedness, tachycardia, and a combination of the preceding, as will be understood by those with skill in the art with respect to this disclosure.

As used in this disclosure, “septic shock” means “sepsis with refractory hypotension,” where the refractory hypotension is characterized by the presence of a persistent systolic blood pressure of less than 90 mm Hg, or a mean arterial blood pressure less than 70 mm Hg, or a reduction of more than 40 mm Hg from baseline in the absence of causes of hypotension other than sepsis despite adequate fluid resuscitation, as will be understood by those with skill in the art with respect to this disclosure.

As used in this disclosure, “multiple organ dysfunction syndrome” (MODS), is equivalent to “multiple organ failure” (MOF), and “multisystem organ failure” (MSOF) and means “septic shock,” where the function of two or more than two organs are altered due to hypoperfusion, hypermetabolism, or both hypoperfusion and hypermetabolism, such that homeostasis cannot be maintained without intervention, and where the two or more than two organs are selected from the group consisting of adrenal glands, brain, gastrointestinal tract, heart, kidneys, liver, lungs, pancreas and spleen, as will be understood by those with skill in the art with respect to this disclosure.

As used in this disclosure, “agent” means a chemical that is active for the treatment of an episode of sepsis, sepsis with septic shock, or sepsis with multiple organ dysfunction syndrome (MODS) in a patient by virtue of raising blood calcium level in the patient, or raising blood calcidiol level in the patient, or raising blood calcitriol level in the patient, as indicated by context.

As used in this disclosure, “composition” means a combination of an agent according to the present invention with either a) one more than one additional agent according to the present invention, or b) one or more than one other chemical that is active for the treatment of an episode of sepsis, sepsis with septic shock, or sepsis with multiple organ dysfunction syndrome (MODS) in a patient by virtue of a mechanism other than raising blood calcium level in the patient, or raising blood calcidiol level in the patient, or raising blood calcitriol level in the patient, or c) both one more than one additional agent according to the present invention and one or more than one other chemical that is active for the treatment of an episode of sepsis, sepsis with septic shock, or sepsis with multiple organ dysfunction syndrome (MODS) in a patient by virtue of a mechanism other than raising blood calcium level in the patient or raising blood calcidiol level in the patient or raising blood calcitriol level in the patient. The composition can further comprise one or more than one chemical that is not active for the treatment of an episode of sepsis, sepsis with septic shock or sepsis with multiple organ dysfunction syndrome (MODS) in the patient, such as for example a binder, a coloring chemical and a flavoring chemical, as will be understood by those with skill in the art with respect to this disclosure.

As used in this disclosure, “functional analog” means a chemical having at least 10% as much biological activity on calcium metabolism or immunomodulation in vivo for an equivalent dose, such as for example a dose by weight or by volume.

As used in this disclosure, “calcidiol,” a prehormone primarily produced in the liver, means (E,3S,6S)-6-[(1R,3aR,4E,7aS)-4-[(2Z)-2-[(5S)-5-hydroxy-2-methylidene-cyclohexylidene]ethylidene]-7a-methyl-2,3,3a,5,6,7-hexahydro-1H-inden-1-yl]-2,3-dimethyl-hept-4-en-2-ol (also known as (6R)-6-[(1R,3aR,4E,7aR)-4-[(2Z)-2-[(5S)-5-Hydroxy-2-methylidene-cyclohexylidene]ethylidene]-7a-methyl-2,3,3a,5,6,7-hexahydro-1H-inden-1-yl]-2-methyl-heptan-2-ol; 25-OH-vitamin D2; 25(OH); vitamin D2; 25(OH)D2; 25(OH)D3; 25-hydroxyvitamin D2; 25-hydroxycalciferol; 25-hydroxycholecalciferol; 25-hydroxyergocalciferol; 25(OH) vitamin D3; 25-hydroxyvitamin D3; calcifediol; and deltalin (Eli Lilly and Company Corp., Indianapolis, Ind., US).

As used in this disclosure, “calcitriol,” the active natural form of vitamin D in a human, means (1R,3S)-5-[2-[(1R,3aR,7aS)-1-[(2R)-6-hydroxy-6-methyl-heptan-2-yl]-7a-methyl-2,3,3a,5,6,7-hexahydro-1H-inden-4-ylidene]ethylidene]-4-methylidene-cyclohexane-1,3-diol (also known as 1-25-dihyroxycholecalciferol; 1,25(OH)2 vitamin D2: 1,25(OH)2 vitamin D3; 1,25-dihydrovitamin D3; 1,25(OH)2D, 1,25(OH)2D3; 1,25-dihydroxycholecalciferol; 1α,25-dihydoxyergocalciferol; 1α,25-dihydroxyvitamin D2; 1α,25-dihydroxyvitamin D3; (1R,3S,5Z)-4-Methylene-5-[(2E)-2-[(1R,3aS,7aR)-octahydro-1-[(1R,2E)-5-hydroxy-1,4,5-trimethyl-2-hexen-1-yl]-7a-methyl-4H-inden-4-ylidene]ethylidene]-1,3-cyclohexanediol; Calcijex® (Abbott Laboratories Corp., Abbott Park, Ill., US); Decostriol (Mibe Jena, Germany, and Jesalis, Hong Kong); ercalcitriol; Rocaltrol® (Validus Pharmaceuticals LLC, Parsippany, N.J., US); Vectical® (Galderma S.A., Cham, Switzerland); and Ro 17-6218).

As used in this disclosure, 24,25-dihydroxycholecalciferol, an inactive form of vitamin D, means (6R)-6-[(1R,3aS,4E,7aR)-4-[(2Z)-2-[(5S)-5-hydroxy-2-methylenecyclohexylidene]ethylidene]-7a-methyl-2,3,3a,5,6,7-hexahydro-1H-inden-1-yl]-2-methylheptane-2,3-diol, (also known as 24,25-dihydroxyvitamin D3; (24R)-hydroxycalcidiol; 24,25(OH)2D); (24R)-hydroxyergocalciferol; and 24(R),25-(OH)2D3).

As used in this disclosure, parathyroid hormone means the 84 amino acids polypeptide that is secreted by the chief cells of the parathyroid glands and that acts to increase the concentration of calcium (Ca2+) in the blood (also known as parathormone; parathyrin; and PTH)

As used in this disclosure, “vitamin D status” means blood levels of the following four chemicals: 1) calcidiol; 2) calcitriol; 3) 24,25-dihydroxycholecalciferol; and 4) parathyroid hormone.

As used in this disclosure, “hypocalcemia blood levels” or “hypocalcemia” means lower than a normal range for the laboratory testing a sample of blood of a patient, as will be understood by those with skill in the art with respect to this disclosure. As an example, hypocalcemia blood levels or hypocalcemia for most laboratories is below 9.0 mg/dL or below 2.2 mmol/L.

As used in this disclosure, “normal blood calcium level” or “normocalcemia” means within a normal range for the laboratory testing a sample of blood of a patient, as will be understood by those with skill in the art with respect to this disclosure. As an example, the normal blood calcium level for most laboratories is between 9.0 and 10.5 mg/dL or between 2.2 and 2.6 mmol/L.

As used in this disclosure, “hypercalcemic blood levels” or “hypercalcemia” means greater than a normal range for the laboratory testing a sample of blood of a patient, as will be understood by those with skill in the art with respect to this disclosure. As an example, hypercalcemic blood levels or hypercalcemia for most laboratories is above 10.5 mg/dL or above 2.6 mmol/L.

As used in this disclosure, “sub-normal blood level,” “normal blood level” and “elevated blood level” of any substance is determined with reference to the normal ranges of the substance for the laboratory testing a sample of blood of a patient, as will be understood by those with skill in the art with respect to this disclosure. As an example, for most laboratories, a sub-normal blood calcidiol level is defined as less than 30 ng/ml, a normal blood calcidiol level is defined as between 30 and 80 ng/ml, and an elevated blood calcidiol level is defined as greater than 80 ng/ml. As another example, for most laboratories, a sub-normal blood calcitriol level is defined as less than 20 pg/ml, and a normal blood calcitriol level is defined as between 20 and 50 pg/ml, and an elevated blood calcitriol level is defined as greater than 50 pg/ml.

According to one embodiment of the present invention, there is provided an agent for the treatment of a patient with an episode of sepsis, sepsis with septic shock, or sepsis with multiple organ dysfunction syndrome. In one embodiment, the agent is selected from the group consisting of 1 alpha-hydroxyergocalciferol; 1-alpha-hydroxyvitamin D2; 1-alpha-hydroxyvitamin D3; 1-alpha(OH)D2; 1-alpha(OH)D3; 1,24,25-trihydroxyvitamin D3; 1,25(OH)2-16-ene-23yne-D3; 19-nor-1,25-dihydroxyvitamin D2); 19-nor-1,25-(OH)2-vitamin D2 (19-nor-1,25-dihydroxyvitamin D2; paricalcitol; Zemplar® (Abbott Laboratories Corp., Abbott Park, Ill. US); 19-nor-1,25-dihydroxyvitamin D3; 20(17→18)-abeo-1α,25-dihydroxy-22-homo-21-norvitamin D(3); 24,25-dihydroxyvitamin D3; alfacalcidiol (1-hydroxycholecalciferol); calcidiol; calcium; calcitriol; cholecalciferol; doxercalciferol; ergocalciferol; EB1089 (24a,26a27a-tri-homo-22,24-diene-1alpha,25(OH)2D3); HM (1,25(OH)2-16-ene-D3); KH1060 (20-epi-22-oxa-24a,26a,27a-tri-homo-1alpha,25(OH)2D3); MC1288 (1,25(OH)2-20-epi-D3) and parathyroid hormone. In another embodiment, the agent is a functional analog of 1 alpha-hydroxyergocalciferol; 1-alpha-hydroxyvitamin D2; 1-alpha-hydroxyvitamin D3; 1-alpha(OH)D2; 1-alpha(OH)D3; 1,24,25-trihydroxyvitamin D3; 1,25(OH)2-16-ene-23yne-D3; 19-nor-1,25-dihydroxyvitamin D2); 19-nor-1,25-(OH)2-vitamin D2 (19-nor-1,25-dihydroxyvitamin D2; paricalcitol; Zemplar® (Abbott Laboratories Corp., Abbott Park, Ill., US); 19-nor-1,25-dihydroxyvitamin D3; 20(17→18)-abeo-1α,25-dihydroxy-22-homo-21-norvitamin D(3); 24 ,25-dihydroxyvitamin D3; alfacalcidiol (1-hydroxycholecalciferol); calcidiol; calcium; calcitriol; cholecalciferol; doxercalciferol; ergocalciferol; EB1089 (24a,26a27a-tri-homo-22,24-diene-1alpha,25(OH)2D3); HM (1,25(OH)2-16-ene-D3); KH1060 (20-epi-22-oxa-24a,26a,27a-tri-homo-1alpha,25(OH)2D3); MC1288 (1,25(OH)2-20-epi-D3) and parathyroid hormone. In a preferred embodiment, the agent is calcitriol. As will be understood by those with skill in the art with respect to this disclosure, the agent can be purified from natural sources or can be synthesized.

According to one embodiment of the present invention, there is provided a composition for the treatment of a patient with an episode of sepsis, sepsis with septic shock, or sepsis with multiple organ dysfunction syndrome. The composition comprises a combination of an agent according to the present invention with either a) one more than one additional agent according to the present invention, or b) one or more than one other chemical that is active for the treatment of an episode of sepsis, sepsis with septic shock, or sepsis with multiple organ dysfunction syndrome (MODS) in a patient by virtue of a mechanism other than raising blood calcium level in the patient, or c) both one more than one additional agent according to the present invention and one or more than one other chemical that is active for the treatment of an episode of sepsis, sepsis with septic shock, or sepsis with multiple organ dysfunction syndrome (MODS) in a patient by virtue of a mechanism other than raising blood calcium level in the patient. The composition can further comprise one or more than one chemical that is not active for the treatment of an episode of sepsis, sepsis with septic shock or sepsis with multiple organ dysfunction syndrome (MODS) in the patient, such as for example a binder, a coloring chemical and a flavoring chemical, as will be understood by those with skill in the art with respect to this disclosure. In a preferred embodiment, the composition comprises calcitriol. In a preferred embodiment, the composition comprises two or more than two agents according to the present invention.

According to one embodiment of the present invention, there is provided a method for treating a patient with an episode of sepsis, where treating comprises either decreasing the severity of an episode of sepsis in the patient, or decreasing the likelihood that the patient with sepsis will progress to sepsis with multiple organ dysfunction syndrome, or decreasing the likelihood that the patient with an episode of sepsis will die from the episode of sepsis, or a combination of the preceding. The method comprises: a) determining that the patient has an episode of sepsis; b) determining a blood calcium level in the patient; c) confirming that the patient has hypocalcemia or normocalcemia but not hypercalcemia; and d) administering to the patient one or more than one dose of an agent or a composition, where the agent is a chemical that raises the blood calcium level in the patient, or the composition comprises an agent that is a chemical that raises the blood calcium level in the patient, thereby treating the patient. In one embodiment, the method further comprises e) determining one or more than one subsequent blood calcium level in the patient; and f) additionally administering to the patient one or more than one dose of the agent or the composition if the one or more than one subsequent blood calcium level in the patient is hypocalcemia or normocalcemia but ceasing the administration of the agent or the composition if the one or more than one subsequent blood calcium level in the patient is hypercalcemia In one embodiment, hypocalcemia is defined as less than 9.0 mg/dL or less than 2.2 mmol/L, normocalcemia is defined as between 9.0 and 10.5 mg/dL or between 2.2 and 2 6 mmol/L, and hypercalcemia is defined as greater than 10.5 mg/dL or greater than 2.6 mmol/L.

According to another embodiment of the present invention, there is provided another method for treating a patient with an episode of sepsis, where treating comprises either decreasing the severity of an episode of sepsis in the patient, or decreasing the likelihood that the patient with sepsis will progress to sepsis with multiple organ dysfunction syndrome, or decreasing the likelihood that the patient with an episode of sepsis will die from the episode of sepsis, or a combination of the preceding. The method comprises: a) determining that the patient has an episode of sepsis; b) determining a first blood calcium level in the patient; c) confirming that the patient has hypocalcemia or normocalcemia but not hypercalcemia; and d) administering to the patient one or more than one dose of an agent or a composition, where the agent is a chemical that raises the blood calcidiol level in the patient, or the composition comprises an agent that is a chemical that raises the blood calcidiol level in the patient, thereby treating the patient; e) determining one or more than one subsequent blood calcium level in the patient; and f) additionally administering to the patient one or more than one dose of the agent or the composition if the one or more than one subsequent blood calcium level in the patient is hypocalcemia or normocalcemia but ceasing the administration of the agent or the composition if the one or more than one subsequent blood calcium level in the patient is hypercalcemia. In one embodiment, hypocalcemia is defined as less than 9.0 mg/dL or less than 2.2 mmol/L, normocalcemia is defined as between 9.0 and 10.5 mg/dL or between 2.2 and 2.6 mmol/L, and hypercalcemia is defined as greater than 10.5 mg/dL or greater than 2.6 mmol/L.

According to another embodiment of the present invention, there is provided another method for treating a patient with an episode of sepsis, where treating comprises either decreasing the severity of an episode of sepsis in the patient, or decreasing the likelihood that the patient with sepsis will progress to sepsis with multiple organ dysfunction syndrome, or decreasing the likelihood that the patient with an episode of sepsis will die from the episode of sepsis, or a combination of the preceding. The method comprises: a) determining that the patient has an episode of sepsis; b) determining a first blood calcium level in the patient; c) confirming that the patient has hypocalcemia or normocalcemia but not hypercalcemia; d) administering to the patient one or more than one dose of an agent or a composition, where the agent is a chemical that raises the blood calcitriol level in the patient, or the composition comprises an agent that is a chemical that raises the blood calcitriol level in the patient, thereby treating the patient; e) determining one or more than one subsequent blood calcium level in the patient; and f) additionally administering to the patient one or more than one dose of the agent or the composition if the one or more than one subsequent blood calcium level in the patient is hypocalcemia or normocalcemia but ceasing the administration of the agent or the composition if the one or more than one subsequent blood calcium level in the patient is hypercalcemia. In one embodiment, hypocalcemia is defined as less than 9.0 mg/dL or less than 2.2 mmol/L, normocalcemia is defined as between 9.0 and 10.5 mg/dL or between 2.2 and 2.6 mmol/L, and hypercalcemia is defined as greater than 10.5 mg/dL or greater than 2.6 mmol/L.

According to another embodiment of the present invention, there is provided another method for treating a patient with an episode of sepsis, where treating comprises either decreasing the severity of an episode of sepsis in the patient, or decreasing the likelihood that the patient with sepsis will progress to sepsis with multiple organ dysfunction syndrome, or decreasing the likelihood that the patient with an episode of sepsis will die from the episode of sepsis, or a combination of the preceding. The method comprises: a) determining that the patient has an episode of sepsis; b) determining a blood calcidiol level in the patient; c) confirming that the patient has a sub-normal blood calcidiol level or a normal blood calcidiol level but not an elevated blood calcidiol level; d) administering to the patient one or more than one dose of an agent or a composition, where the agent is a chemical that raises the blood calcidiol level in the patient, or the composition comprises an agent that is a chemical that raises the blood calcidiol level in the patient, thereby treating the patient; e) determining one or more than one subsequent blood calcidiol level in the patient; and f) additionally administering to the patient one or more than one dose of the agent or the composition if the one or more than one subsequent blood calcidiol level in the patient is sub-normal blood calcidiol level or normal blood calcidiol level but ceasing the administration of the agent or the composition if the one or more than one subsequent blood calcidiol level in the patient is elevated blood calcidiol level. In one embodiment, the sub-normal blood calcidiol level is defined as less than 30 ng/ml, the normal blood calcidiol level is defined as between 30 and 80 ng/ml, and the elevated blood calcidiol level is defined as greater than 80 ng/ml.

According to another embodiment of the present invention, there is provided another method for treating a patient with an episode of sepsis, where treating comprises either decreasing the severity of an episode of sepsis in the patient, or decreasing the likelihood that the patient with sepsis will progress to sepsis with multiple organ dysfunction syndrome, or decreasing the likelihood that the patient with an episode of sepsis will die from the episode of sepsis, or a combination of the preceding. The method comprises: a) determining that the patient has an episode of sepsis; b) determining a blood calcitriol level in the patient; c) confirming that the patient has a sub-normal blood calcitriol level or a normal blood calcitriol level but not an elevated blood calcitriol level; and d) additionally administering to the patient one or more than one dose of the agent or the composition if the one or more than one subsequent blood calcitriol level in the patient is sub-normal blood calcidiol level or normal blood calcidiol level but ceasing the administration of the agent or the composition if the one or more than one subsequent blood calcitriol level in the patient is elevated blood calcidiol level. In one embodiment, the sub-normal blood calcitriol level is defined as less than 20 pg/ml, the normal blood calcitriol level is defined as between 20 and 50 pg/ml, and the elevated blood calcitriol level is defined as greater than 50 ng/ml.

In one embodiment of the method for treating a patient with an episode of sepsis, the one or more than one dose administered is between 0.01 ng and 1 gram. In another embodiment, the one or more than one dose administered is between 0.01 ng and 10,000 mcg. In another embodiment, the one or more than one dose administered is between 1 ng and 10,000 mcg. In another embodiment, the one or more than one dose administered is between 0.1 mcg and 10,000 mcg. In another embodiment, the one or more than one dose administered is between 1 mcg and 1,000 mcg. In another embodiment, the one or more than one dose administered is between 100 units to 10,000,000 units. In another embodiment, the one or more than one dose administered is between 400 units to 10,000,000 units. In another embodiment, the one or more than one dose administered is between 1000 units to 10,000,000 units. In another embodiment, the one or more than one dose administered is between 1000 units to 1,000,000 units. In another embodiment, the one or more than one dose administered is 1000 units to 100,000 units. In another embodiment, the one or more than one dose administered is between 1000 units to 10,000 units. In another embodiment, the one or more than one dose is administered by a route selected from the group consisting of intrarectal, intramuscular, intraperitoneal, intrathecal, intravenous and oral. In another embodiment, the one or more than one dose is a plurality of doses. In another embodiment, the plurality of doses is two doses. In another embodiment, the plurality of doses is three doses. In another embodiment, the plurality of doses is four doses. In another embodiment, the plurality of doses is more than four doses. In another embodiment, the plurality of doses is twenty-eight doses. In another embodiment, the plurality of doses are administered between one hour and seventy-two hours apart. In another embodiment, the plurality of doses are administered between one hour and thirty-six hours apart. In another embodiment, the plurality of doses are administered between one hour and twenty-four hours apart. In another embodiment, the plurality of doses are administered twenty-four hours apart. In another embodiment, the plurality of doses are 2 mcg of paricalcitol administered once daily for twenty-eight days. In another embodiment, the agent is selected from the group consisting of 1 alpha-hydroxyergocalciferol; 1-alpha-hydroxyvitamin D2; 1-alpha-hydroxyvitamin D3; 1-alpha(OH)D2; 1-alpha(OH)D3; 1,24,25-trihydroxyvitamin D3; 1,25(OH)2-16-ene-23yne-D3; 19-nor-1,25-dihydroxyvitamin D2; 19-nor-1,25-(OH)2-vitamin D2; 19-nor-1,25-dihydroxyvitamin D3; 20(17→18)-abeo-1α,25-dihydroxy-22-homo-21-norvitamin D(3); 24,25-dihydroxyvitamin D3; alfacalcidiol; calcidiol; calcium; calcitriol; cholecalciferol; doxercalciferol; ergocalciferol; EB1089; HM; KH1060; MC1288 and parathyroid hormone. In another embodiment, the agent is a functional analog of one or more than one of the group of 1 alpha-hydroxyergocalciferol; 1-alpha-hydroxyvitamin D2; 1-alpha-hydroxyvitamin D3; 1-alpha(OH)D2; 1-alpha(OH)D3; 1,24,25-trihydroxyvitamin D3; 1,25(OH)2-16-ene-23yne-D3; 19-nor-1,25-dihydroxyvitamin D2; 19-nor-1,25-(OH)2-vitamin D2; 19-nor-1,25-dihydroxyvitamin D3; 20(17→18)-abeo-1α,25-dihydroxy-22-homo-21-norvitamin D(3); 24,25-dihydroxyvitamin D3; alfacalcidiol; calcidiol; calcium; calcitriol; cholecalciferol; doxercalciferol; ergocalciferol; EB1089; HM; KH1060; MC1288 and parathyroid hormone. In another embodiment, the agent is calcitriol. In another embodiment, the composition comprises at least two agents. In another embodiment, the composition comprises at least two agents selected from the group consisting of 1 alpha-hydroxyergocalciferol; 1-alpha-hydroxyvitamin D2; 1-alpha-hydroxyvitamin D3; 1-alpha(OH)D2; 1-alpha(OH)D3; 1,24,25-trihydroxyvitamin D3; 1,25(OH)2-16-ene-23yne-D3; 19-nor-1,25-dihydroxyvitamin D2; 19-nor-1,25-(OH)2-vitamin D2; 19-nor-1,25-dihydroxyvitamin D3; 20(17→18)-abeo-1α,25-dihydroxy-22-homo-21-norvitamin D(3); 24,25-dihydroxyvitamin D3; alfacalcidiol; calcidiol; calcium; calcitriol; cholecalciferol; doxercalciferol; ergocalciferol; EB1089; HM; KH1060; MC1288, parathyroid hormone, and a functional equivalent of 1-alpha-hydroxyvitamin D2; 1-alpha-hydroxyvitamin D3; 1-alpha(OH)D2; 1-alpha(OH)D3; 1,24,25-trihydroxyvitamin D3; 1,25(OH)2-16-ene-23yne-D3; 19-nor-1,25-dihydroxyvitamin D2; 19-nor-1,25-(OH)2-vitamin D2; 19-nor-1,25-dihydroxyvitamin D3; 20(17→18)-abeo-1α,25-dihydroxy-22-homo-21-norvitamin D(3); 24,25-dihydroxyvitamin D3; alfacalcidiol; calcidiol; calcium; calcitriol; cholecalciferol; doxercalciferol; ergocalciferol; EB1089; HM; KH1060; MC1288, and parathyroid hormone, and a functional analog of the preceding.

According to another embodiment of the present invention, there is provided a method for characterizing the severity of an episode of sepsis in a patient, by determining a likelihood that a patient with an episode of sepsis will progress to sepsis with multiple organ dysfunction syndrome (MODS) (“multiple organ failure” (MOF), and “multisystem organ failure” (MSOF)), or by determining a likelihood that a patient with an episode of sepsis will die from the episode of sepsis, where the method comprises a) determining that the patient has an episode of sepsis, and b) determining a blood level of calcium in the patient, where a blood level of calcium below a predetermined amount indicates an increased likelihood that a patient with an episode of sepsis will progress to sepsis with multiple organ dysfunction syndrome, or that the patient will die from the episode of sepsis. In one embodiment, the predetermined blood level of calcium is less than 9.0 mg/dL or less than 2.2 mmol/L. According to another embodiment of the present invention, there is provided a method for characterizing the severity of an episode of sepsis in a patient, by determining a likelihood that a patient with an episode of sepsis will progress to sepsis with multiple organ dysfunction syndrome (MODS) (“multiple organ failure” (MOF), and “multisystem organ failure” (MSOF)), or by determining a likelihood that a patient with an episode of sepsis will die from the episode of sepsis, where the method comprises a) determining that the patient has an episode of sepsis, and b) determining a blood level of calcidiol in the patient, where a blood level of calcidiol below a predetermined amount indicates an increased likelihood that a patient with an episode of sepsis will progress to sepsis with multiple organ dysfunction syndrome, or that the patient will die from the episode of sepsis. In one embodiment, the predetermined blood level of calcidiol is less than 30 ng/ml. According to another embodiment of the present invention, there is provided a method for characterizing the severity of an episode of sepsis in a patient, by determining a likelihood that a patient with an episode of sepsis will progress to sepsis with multiple organ dysfunction syndrome (MODS) (“multiple organ failure” (MOF), and “multisystem organ failure” (MSOF)), or by determining a likelihood that a patient with an episode of sepsis will die from the episode of sepsis, where the method comprises a) determining that the patient has an episode of sepsis, and b) determining a blood level of calcitriol in the patient, where a blood level of calcitriol below a predetermined amount indicates an increased likelihood that a patient with an episode of sepsis will progress to sepsis with multiple organ dysfunction syndrome, or that the patient will die from the episode of sepsis. In one embodiment, the predetermined blood level of calcitriol is less 30 pg/mL. In another embodiment, the predetermined blood level of calcitriol is less than 25 pg/mL. In another embodiment, the predetermined blood level of calcitriol is less than 20 pg/mL. In another embodiment, the predetermined blood level of calcitriol is less than 15 pg/mL. In another embodiment, the predetermined blood level of calcitriol is less than 14 pg/mL. In another embodiment, the predetermined blood level of calcitriol is less than 13.6 pg/mL. In another embodiment, the predetermined blood level of calcitriol is less than 10 pg/mL. In another embodiment, the predetermined blood level of calcitriol is less than 5 pg/mL. In one embodiment, identifying the patient comprises reference to medical records relevant to the patient. In another embodiment, identifying the patient comprises diagnosing the episode of sepsis in the patient. In a preferred embodiment, diagnosing the episode of sepsis in the patient comprises determining that the patient has the following criteria: 1) either i) a suspected or confirmed source of infection determined by the treating clinician, or ii) a serum lactate level greater than 2.5 mmol/L or both; and 2) two or more criteria selected from the group consisting of: a) a body temperature greater than 38° C. or less than 36° C.; b) i) a respiratory rate greater than 20 breaths per minute, or ii) partial pressure of carbon dioxide less than 32 mm Hg; c) a heart rate greater than 90 beats per minute; and d) i) a white blood cell count greater than 12,000 cells per mm³, or ii) a white blood cell count less than 4,000 cells per mm³, or iii) a white blood cell count comprising 10% or more than 10% immature forms. In one embodiment, measuring the blood level in the patient comprises measuring the blood level in the patient a plurality of times. In a preferred embodiment, the plurality of times is two times. In another preferred embodiment, the plurality of times is three times. In another preferred embodiment, the plurality of times is four times. In another preferred embodiment, the plurality of times is more than four times. In one embodiment, the blood level in the patient is measured upon admission to a health care facility. In another embodiment, the blood level is measured upon diagnosing the patient with the episode of sepsis. In one embodiment, the calcitriol blood level is measured at one or more than one time selected from the group consisting of 0, 24, 48, 72 and 96 hours after admission to a health care facility. In another embodiment, the blood level is measured at one or more than one time selected from the group consisting of 0, 24, 48, 72 and 96 hours after diagnosing the patient with the episode of sepsis. In one embodiment, the method further comprises determining a presence of one or more than one patient criterion in the patient selected from the group consisting of age, Acute Physiology and Chronic Health Evaluation (APACHE) II scores, and a presence of stroke comorbidity, and where the presence of one or more than one criterion of an age greater than 70 years, an Acute Physiology and Chronic Health Evaluation II score greater than 25, and stroke comorbidity further increases the likelihood that a patient with an episode of sepsis will progress to sepsis with multiple organ dysfunction syndrome, or that the patient will die from the episode of sepsis.

### EXAMPLE I

### Determination of Vitamin D Status in Patients with Sepsis

According to the present invention, vitamin D status was determined in patients with sepsis as follows, where vitamin D status was defined as blood levels of calcidiol, calcitriol, 24,25-dihydroxycholecalciferol and parathyroid hormone. An analysis of stored plasma samples was conducted, where the stored plasma samples were taken from ninety-one patients diagnosed with sepsis that were admitted to the Medical Intensive Care Unit at Loma Linda University Medical Center (Loma Linda, Calif., US). All patients were aged 18 years or older and met the following criteria for a diagnosis of sepsis: 1) either i) a suspected or confirmed source of infection determined by the treating clinician, or ii) a serum lactate level greater than 2.5 mmol/L or both; and 2) two or more criteria selected from the group consisting of: a) a body temperature greater than 38° C. or less than 36° C.; b) i) a respiratory rate greater than 20 breaths per minute, or ii) partial pressure of carbon dioxide less than 32 mm Hg; c) a heart rate greater than 90 beats per minute; and d) i) a white blood cell count greater than 12,000 cells per mm³, or ii) a white blood cell count less than 4,000 cells per mm³, or iii) a white blood cell count comprising 10% or more than 10% immature forms. Patients were excluded if they experienced cardiac arrest on arrival to the facility, were under a do-not-attempt-resuscitation order, or were pregnant.

Baseline patient characteristics were obtained at admission, including Acute Physiology and Chronic Health Evaluation (APACHE) II scores, comorbidities, demographics, routine laboratories including cultures, and notation of any suspected source of infection. Additionally, serial measurements of vitamin D status were made at hours 0, 24, 48, 72 after admission. The primary outcome measurement was recorded as patient nonsurvival (mortality) at thirty days after admission, which was considered synonymous with multiple organ dysfunction syndrome.

To determine vitamin D status, whole blood was collected at hours 0, 24, 48, and 72 after admission by venipuncture into collection tubes containing ethylenediaminetetraacetic acid (EDTA) as an anticoagulant. Within one hour of collection, each sample was centrifuged at 2,000×g for ten minutes. The plasma was immediately aliquoted into 1 mL cryovials and stored at −84° C. without further freeze-thaw cycles. One mL of each plasma sample was shipped in dry ice to Heartland Assay, LLC (Ames, Iowa, US) for analysis of calcidiol, calcitriol, 24,25-dihydroxycholecalciferol and parathyroid hormone blood levels.

Calcidiol blood levels were measured according to standard techniques using a Food and Drug Administration (FDA, US) approved direct, competitive chemiluminescence immunoassay (CLIA) using a DiaSorin LIAISON® 25-OH Vitamin D Total Assay (Diasorin S.p.A., 13040 Saluggia (Vercelli) IT). This assay is co-specific for calcidiol and calcitriol, and utilizes a specific antibody to calcidiol coating magnetic particles (solid phase) and a vitamin D analogue, 22-carboxy-23,24,25,26,27-pentanorvitamin D₃, linked to an isoluminol derivative. During the incubation, calcidiol is dissociated from its binding protein, and competes with the isoluminol labeled analogue for binding sites on the antibody. After the incubation, the unbound material is removed with a wash cycle. Subsequently, the starter reagents are added and a flash chemiluminescent reaction is initiated. The light signal is measured by a photomultiplier as relative light units (RLU) and is inversely proportional to the concentration of calcidiol present in a calibrator, a control or a sample of interest. The assay has a normal range of between 30 ng/mL and 80 ng/mL, with inter- and intra-assay coefficients of variability of 11.2% and 8.1%, respectively.

Calcitriol blood levels were measured according to standard techniques using an assay that extracts and purifies vitamin D metabolites from serum or plasma using C18OH cartridges. Following extraction, the sample of interest is assayed using a competitive radioimmunoassay (RIA) procedure based on a polyclonal antibody that is specific for both ercalcitriol and calcidiol. The sample of interest, antibody and tracer are incubated for two hours at between 20-25° C. Phase separation is accomplished after twenty minutes of incubation at 20-25° C. with a second antibody precipitating complex. After centrifugation and decantation, the bound fraction remaining in the pellet is counted in a gamma counter. Values are calculated directly from a calibrator curve of known concentrations. The assay has a normal range of between 20 pg/mL and 50 pg/mL, with inter- and intra-assay coefficients of variability of 12.6% and 9.8%, respectively.

24,25-dihydroxycholecalciferol blood levels were measured by a modification of the procedure described by Horst et al. Briefly, plasma lipids were extracted and purified, first, by silica solid phase extraction (SPE), and then, by high pressure liquid chromatography (HPLC). The HPLC purified material which contained both 24,25(OH)2D2 and 24,25(OH)2D3 was assayed by radioimmunoassay (RIA). [3H]-24,25(OH)2D3 was used to estimate losses. The RIA method was based on an antibody which was co-specific for 24,25-dihydroxycholecalciferol and 24,25(OH)2D2. The sample of interest, antibody and tracer are incubated for 120 minutes at 20-25° C. Phase separation is accomplished after twenty minutes incubation at 20-25° C. with a second antibody precipitating complex. A NSB/Addition buffer is added after this incubation prior to centrifugation to aid in reducing non-specific binding. Radioactivity was quantitated by the γ-radiation counting system with use of a smooth-spline method. The assay has a normal range of between 1 ng/mL and 3 ng/mL, with inter- and intra-assay coefficients of variability of 10.0% and 8.0%, respectively.

Parathyroid hormone blood levels were measured according to standard techniques using a Food and Drug Administration (FDA, US) approved DiaSorin's N-tact® PTH SP immunoradiometric assay (IRMA). This assay utilizes two different polyclonal antibodies that have been purified using affinity chromatography. These purified antibodies are specific for two different regions of the parathyroid hormone molecule. The first antibody, is specific for parathyroid hormone amino acids 39-84 and is bound to a solid phase (polystyrene bead). The second antibody is specific for parathyroid hormone amino acids 1-34 and is labeled with iodine-125. The sample of interest is incubated simultaneously with both antibodies. Intact parathyroid hormone 1-84 contains both the 1-34 and 39-84 amino acid sequences and is the only form of parathyroid hormone that will be bound to both the antibody on the bead and the antibody labeled with iodine-125. Since the antibody coupled to the solid phase is specific for C-terminal and mid-region fragments as well as intact parathyroid hormone, the capacity of the solid phase is designed to accommodate very high levels of parathyroid hormone. This prevents interference by extremely elevated C-terminal and mid-region parathyroid hormone fragments in samples. Following the incubation period, each bead is washed to remove any unbound labeled antibody. The radioactivity present in the remaining bound labeled antibody is then measured using a gamma counter. The concentration of intact parathyroid hormone in the sample of interest is directly proportional to the radioactivity measured. The assay has a normal range of between 13.6 pg/mL and 65 pg/mL, with inter- and intra-assay coefficients of variability of 4.3% and 2.7%, respectively.

### EXAMPLE II

### Analysis of Relationship Between Baseline Characteristics, Patient Survival and Patient Nonsurvival at Thirty Days from Admission in Patients with Sepsis

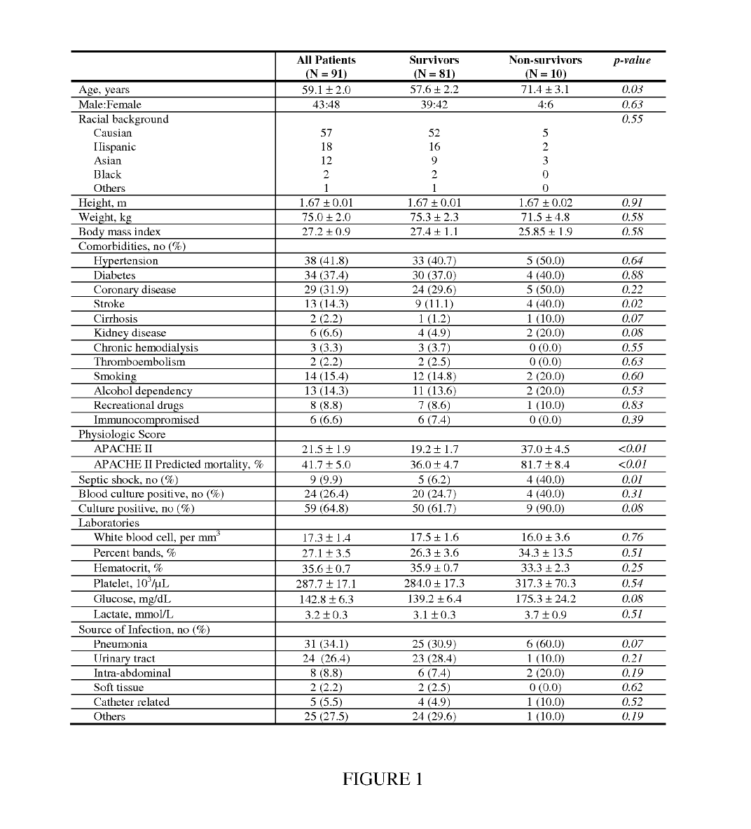

The baseline patient characteristics were analyzed to determine their relationship with patient survival and patient nonsurvival at thirty days from admission with sepsis by comparing the baseline patient characteristics between patient survivors and patient non-survivors using the student's T-test for normally distributed continuous variables, and the Mann-Whitney U test for skewed variables. The chi-squared test was used for categorical variables. Referring now to FIG. 1, there is shown a table of baseline patient characteristics in patients studied according to Example I relating the baseline patient characteristics to patient survival or patient nonsurvival. The data are presented as mean±standard error, or count (a percentage of the column)

### EXAMPLE III

### Analysis of Relationship Between Vitamin D Status and Other Potentially Relevant Characteristics, and Patient Nonsurvival at Thirty Days from Admission in Patients with Sepsis

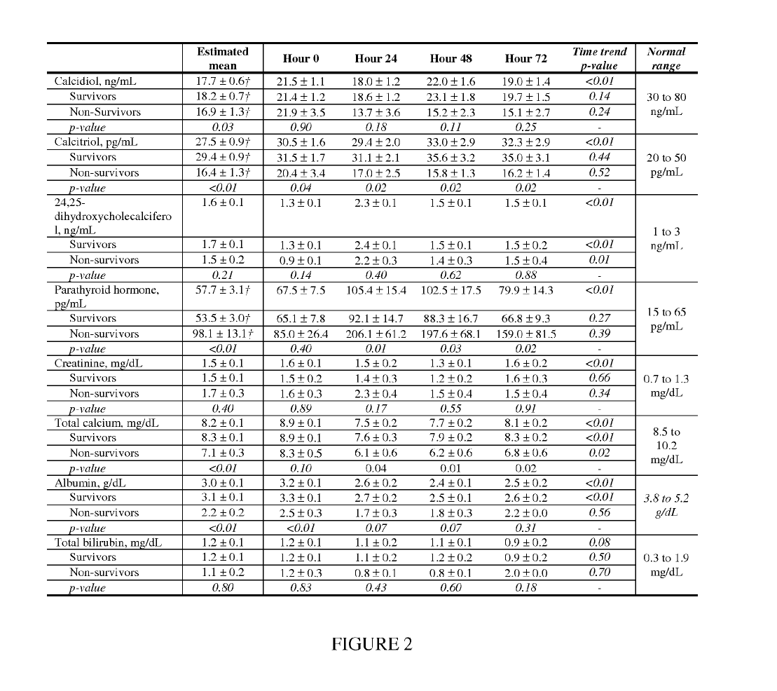

The vitamin D status and other potentially relevant characteristics were analyzed to determine their relationship with patient survival and patient nonsurvival at thirty days from admission with sepsis. A Time variable was created to represent repeated measurements over hours 0, 24, 48, and 72. The Time variable was utilized in repeated measures analysis of variance (ANOVA), which was performed on the vitamin D status characteristics to test for the significance of changes in values over time and to obtain least square estimated group means. Natural log transformations were required for variables that violate the assumptions of linear regression. Referring now to FIG. 2, there is shown a table of vitamin D status characteristics, and of other potentially relevant characteristics (creatinine, total calcium, albumin, and total bilirubin blood levels) measured at 0, 24, 48, and 72 hours from admission, and analyzed to determine their predictive value for patient survival and patient nonsurvival at thirty days from admission for sepsis. The Time trend p-value is the change in the variable over the four measurement periods derived from a repeated measure ANOVA. The overall estimated mean is the least-square mean derived from a repeated measure ANOVA. The results indicated by the t reflect analysis of the natural log transformed variables. The data are presented as mean±standard error.

### EXAMPLE IV

### Analysis of Predictive Value of Variables Found to Have Potential Relevance to Patient Survival and Patient Nonsurvival at Thirty Days from Admission with Sepsis

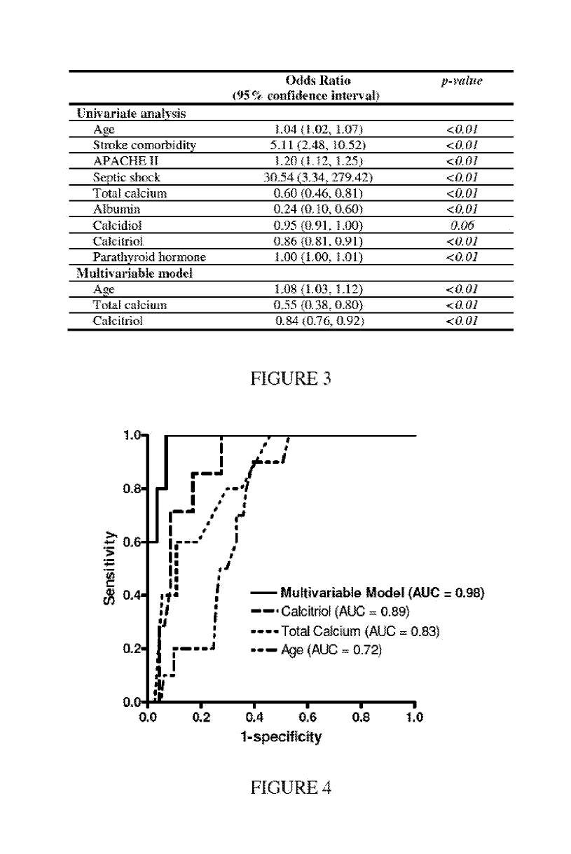

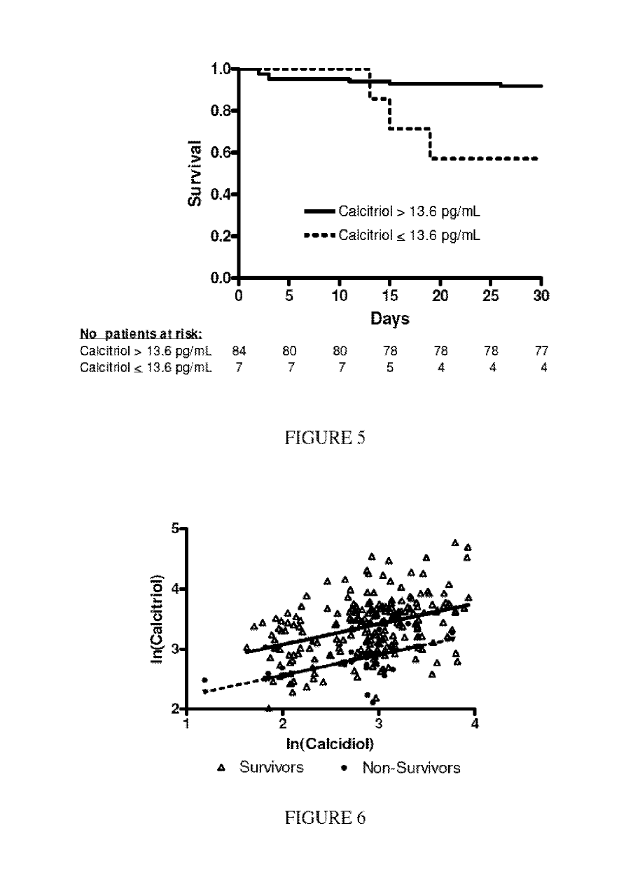

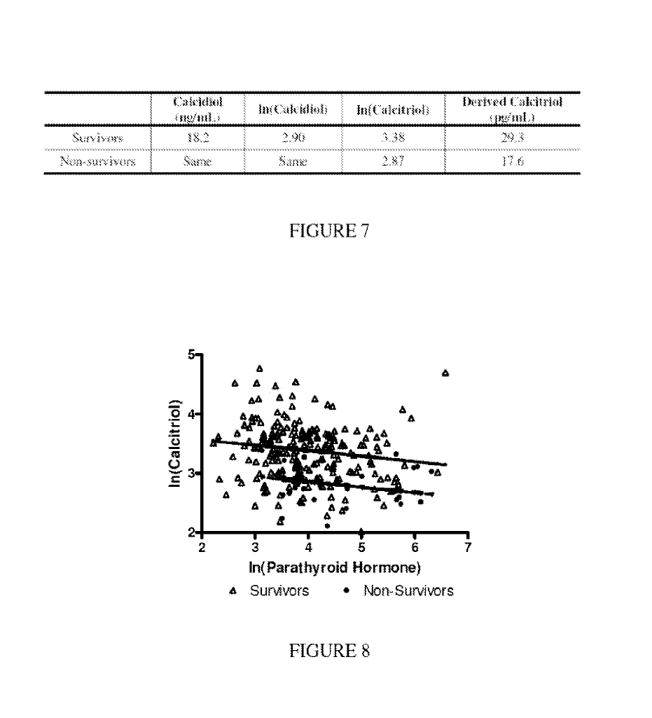

Next, the predictive value of variables found to have potential relevance to patient survival and patient nonsurvival at thirty days from admission with sepsis were analyzed. First, a univariate analysis was performed on variables that were associated with patient survival and patient nonsurvival at thirty days from admission with sepsis as shown in FIG. 2. To obtain odds ratios, an analysis of longitudinal data using the Generalized Estimating Equations method (GEE) was then performed only on those variables that were significant in the univariate analysis. The final multivariable model was derived by forcing age into the model to test the main effect of calcitriol blood levels on outcome, and adding one at a time those variables in the univariate analysis that were significantly associated with the outcome variable. Significance as well as goodness-of-fit statistics was considered when selecting the appropriate final model. The transformation was not applied since GEE method of analysis is robust against assumption of homogeneity of variance. A Time variable reflecting repeated measurements over hours 0, 24, 48, and 72, was included and did not affect the results of the model at α=0.05. Nine patients in this analysis developed multiple organ failure (MOF), resulting in a large 95% confidence interval for multiple organ failure (MOF) as a predictor of mortality. Referring now to FIG. 3, there is shown a table of the predictive value of variables found to have potential relevance to patient survival and patient nonsurvival at thirty days from admission with sepsis. Referring now to FIG. 4, there is shown a graph of receiver operating characteristics (ROC) curves that were generated to determine the predictive value of age, total calcium and calcitriol blood levels at 48 hours after admission with respect to patient survival and patient nonsurvival at thirty days from admission with sepsis, where the area under the receiver operating characteristics curve is AUC. Referring now to FIG. 5, there is shown a graph of Kaplan-Meir analysis performed to obtain patient survival curves over thirty days from admission for calcitriol blood levels, the variable with the highest area under the receiver operating characteristics curve, showing that patients estimated mean calcitriol blood levels<13.6 pg/mL over 72 hours had a mean survival time of 17.6 days, while patients with calcitriol blood levels 13.6 pg/mL had a mean survival time of 24.6 days. Referring now to FIG. 6 and FIG. 7, there are shown, respectively, a graph of the natural log of calcitriol blood levels as a function of the natural log of calcidiol blood levels for all patients (FIG. 6); and a table of calcidiol blood levels and calcitriol blood levels in patient survivors and patient non-survivors from repeated measures regression analysis of the values in FIG. 6, showing that calcitriol production was lower in non-survivors for the same substrate calcidiol blood levels in survivors (FIG. 7). As can be seen, in both patient survivors and patient non-survivors, there was a significant increase in ln(calcitriol) for increases in ln(calcidiol) (p<0.01 and p=0.02, respectively), such that ln(calcitriol)=2.39+0.34(ln(calcidiol)) for survivors, and 1.88+0.34(ln(calcidiol)) for non-survivors. For the same blood levels of calcidiol, blood levels of calcitriol would be lower in non-survivors compared to survivors (p<0.01). Finally, referring now to FIG. 8, there is shown a graph of the natural log of calcitriol blood levels as a function of the natural log of parathyroid hormone (PTH) blood levels for all patients. As can be seen, both patient survivors and patient non-survivors have a parallel decrease in ln(calcitriol) blood levels for increases in ln(parathyroid hormone) blood levels. The relationship was statistically significant (p<0.01) in the patient survivors. The relationship was not statistically significant in the patient non-survivors, possibly due to small number of patient non-survivors, however, a trend was clearly apparent (p=0.80).

### EXAMPLE V

### Conclusions of Predictive Value of Variables Found to Have Potential Relevance to Patient Survival and Patient Nonsurvival at Thirty Days from Admission with Sepsis

Using the analyses performed in Example II, Example III and Example IV, the following conclusions were made regarding the predictive value of variables found to have potential relevance to patient survival and patient nonsurvival at thirty days from admission with sepsis. All analyses were performed using PASW 18.0 (SPSS, Inc., Chicago, Ill, US) or SAS 9.3 (SAS Institute, Inc., Cary, N.C., US). Statistical significance was defined at p<0.05 (α=0.05). At admission, there were significant differences in age, APACHE II, a presence of septic shock and a presence of stroke comorbidity, between patient survivors and patient non-survivors as shown in FIG. 1. At hour 0 after admission, albumin and calcitriol blood levels were also significantly different between survivors and non-survivors as shown n FIG. 2. Further, at hour 0 after admission, all patients met criteria for vitamin D insufficiency (i.e., calcidiol blood level<30 ng/mL), but there was no significant difference between patient survivors and patient non-survivors in calcidiol, 24,25-dihydroxycholecalciferol or parathyroid hormone blood levels as shown in FIG. 2. Calcitriol blood levels were different between the patient survivors and patient non-survivors hour 0 after admission, 31.5+1.7 vs. 20.4+3.4 pg/mL in patient survivors vs. patient non-survivors, respectively (p=0.04) as seen in FIG. 2. In contrast to the hour 0 after admission values, the estimated mean over the 72-hour period after admission for albumin, calcidiol, calcitriol, parathyroid hormone and total calcium blood levels were significantly different between patient survivors and patient non-survivors, while there was no difference in 24,25-dihydroxycholecalciferol, creatinine or total bilirubin blood levels as seen in FIG. 2.

The univariate analysis as shown in FIG. 3 demonstrated that age, APACHE II, stroke comorbidity, and septic shock between patient survivors and patient non-survivors, were associated with increased likelihood for patient nonsurvival; whereas increased albumin, calcitriol, and total calcium blood levels were associated with decreased likelihood for mortality. There was no significant difference in likelihood of mortality for calcidiol blood levels. Finally, the multivariable model showed that age, total calcium, and calcitriol were independently associated with mortality at thirty days after admission, with odds ratio (95% confidence interval) of 1.08 (1.03,1.12), 0.55 (0.38,0.80), and 0.84 (0.76, 0.92), respectively.

Additionally, area under the curve (AUC) of the receiver operating characteristics (ROC) curve for calcitriol blood levels measured at hours 0, 24, 48, and 72 for discriminating thirty day mortality were 0.74, 0.85, 0.89, and 0.87, respectively. The area under the curve of the receiver operating characteristics curve for total calcium measured at hours 0, 24, 48, and 72 were 0.65, 0.76, 0.83, and 0.81, respectively. The area under the curve of the receiver operating characteristics curve for age were 0.72. Since the area under the curve for both calcitriol and total calcium showed the highest area under the curve at hour 48, this time point was chosen for determining the receiver operating characteristics curve of the combined model. The area under the curve of the receiver operating characteristics of the combined model including age, total calcium, and calcitriol at hour 48 was 0.98 as shown in FIG. 4. Further analysis showed that a cutoff in estimated mean calcitriol measured over 72 hours of <13.6 pg/mL had the best accuracy (or maximum sum of true positive and true negative) for predicting patient survival or patient nonsurvival at thirty days after admission. Patients with calcitriol blood levels<13.6 pg/mL had 57.1% thirty day survival, compared to 91.7% thirty day survival in patients with calcitriol blood levels>13.6 pg/mL. The mean survival time for patients with estimated mean calcitriol blood levels<13.6 pg/mL measured over 72 hours was 17.6 days, compared to 24.6 days in patients with calcitriol blood levels>13.6 pg/mL (p<0.01) as shown in FIG. 5.

Repeated measures regression analysis of natural log (ln) of calcitriol blood levels as a function of ln(calcidiol) showed that calcitriol is positively dependent on calcidiol blood levels in both patient survivors and patient non-survivors (p<0.01 and p=0.02, respectively) as seen in FIG. 6. However, based on the regression equations, ln(calcitriol)=2.39+0.34(ln(calcidiol)) for patient survivors and 1.88+0.34(ln(25(OH)D)) for patient non-survivors, the same amount of substrate calcidiol would result in lower calcitriol in non-survivors compared to patient survivors (p<0.01) as seen in FIG. 6 and FIG. 7. Analysis of ln(calcitriol) as a function of ln(parathyroid hormone) showed that increased levels of parathyroid hormone did not increase calcitriol. Instead in both patient survivors and patient non-survivors, there was a parallel decrease in calcitriol with increasing parathyroid hormone blood levels (p<0.01 and p=0.80, respectively) as seen in FIG. 8.

Therefore, as can be seen, calcitriol blood levels have a strong predictive value for patient survival and patient nonsurvival at thirty days from admission with sepsis, while calcidiol blood levels were not strongly predictive value for patient survival and patient nonsurvival at thirty days from admission with sepsis. Additionally, estimated mean calcitriol blood levels<13.6 pg/mL measured over 72 hours after admission had the strongest predictive value for patient nonsurvival at thirty days from admission with sepsis from progression to multiple organ dysfunction syndrome, while calcitriol blood levels≧13.6 pg/mL over 72 hours after admission had the strongest predictive value for patient survival at thirty days from admission with an episode of sepsis. These findings appear to be unique to this disclosure.

The data and analyses also indicate that the decreased blood levels of calcitriol in patient non-survivors were not due to degradation of calcitriol because 24-hydroxylase activity measured by serum 24,25-dihydroxycholecalciferol levels were in the normal range and not significantly different between patient survivors and patient non-survivors. Therefore, the decreased blood levels of calcitriol in patient non-survivors were likely due to decreased production. The production of calcitriol in the blood is determined from the conversion of the calcidiol by the renal enzyme 1α-hydroxylase. There was no significant difference in the calcidiol blood levels between patient survivors and patient non-survivors at hour 0 after admission. However, over the 72-hour study period, the estimated mean of calcidiol was significantly lower in the patient non-survivors, and could reduce the amount of calcitriol produced. Although calcidiol blood levels were lower in patient non-survivors, the relationship between calcidiol blood levels and calcitriol blood levels (i.e., the slope of ln(calcitriol) as a function of ln(calcidiol) (FIG. 6) was equivalent in both patient survivors and patient non-survivors. However, the lower calcidiol blood levels in patient non-survivors were insufficient to account for the larger decrease in calcitriol blood levels such that for the same calcidiol blood level, there would be a significantly lower calcitriol blood level in the patient non-survivors compared to patient survivors. This indicates that there is some fundamental aberrancy in the renal 1α-hydroxylase enzyme in sepsis that the aberrancy is greater in patient non-survivors than in patient survivors. If volume resuscitation during the treatment course of sepsis had impacted vitamin D levels, then similar decreases in calcitriol would have been expected in both survivors and non-survivors based on 25(OH)D levels. There was no difference in the amount of fluids given during the 72-hour study period in survivors compared to non-survivors, 6,024+544 vs. 8,375+870 mL (p=0.19), but the non-survivors had a 40% lower calcitriol for the same level of calcidiol as in the survivors (FIG. 7. Further, one of the main regulators of the renal 1α-hydroxylase enzyme activity is serum parathyroid hormone. Parathyroid hormone blood levels were increased in both patient survivors and patient non-survivors, but more in patient non-survivors. Generally, an increase in parathyroid hormone would be accompanied by increased calcitriol, as seen in hyperparathyroidism. Contrary to the expected positive correlation between parathyroid hormone blood levels and calcitriol blood levels, however, there was a negative correlation, that is, calcitriol blood levels decreased at high levels of parathyroid hormone blood levels in both patient survivors and patient non-survivors. Additionally, for each parathyroid hormone blood level, there appeared to be lower calcitriol production in patient non-survivors compared to patient survivors. These unexpected findings indicate that renal 1α-hydroxylase enzyme activity in sepsis is insensitive to parathyroid hormone, its major regulator. Taken together, these findings indicate that there are two abnormalities in renal 1α-hydroxylase enzyme activity in sepsis patients. First, there is insensitivity to parathyroid hormone stimulation of calcitriol production which applies to both patient survivors and patient non-survivors. Second, there is an additional abnormality in the patient non-survivors, which accounts for the additional significantly lower calcitriol blood levels that are independent of the calcidiol blood levels. As can now be appreciated, both renal 1α-hydroxylase enzyme and the endocrine production of calcitriol is pivotally disturbed in sepsis, leading to a dysfunction of calcitriol action throughout the body, including in white blood cells and epithelial cells which plays a critical role in immunity, containing “leaky” vasculature and an increase in cytokine production associated with sepsis, and regulating cathelicidin which acts to kill invading microbes. Additionally, the analyses also indicate that total calcium blood levels were significantly decreased in the patient non-survivors compared with the patient survivors.

### EXAMPLE VI

### Determination of Optimal Dosing of Calcitriol for Treatment of Sepsis

Then, the optimal dosing of calcitriol as an example of an agent for treatment of sepsis according to the present invention was determined. Anesthesia was induced in adult Wistar rats with 5% isoflurane followed by maintenance on 2.5% isoflurane. The cecum was exteriorized via a small abdominal incision, and was then ligated approximately 3.5 connection modules 12 distal to the cecum tip. The ligated cecum was punctured twice with a 26-gauge needle and was gently squeezed to extrude some stool. The cecum was replaced in the abdomen and the incision closed with 4-0 silk sutures in two layers, abdominal wall and skin. This Cecum Ligation-Puncture (CLP) procedure induced sepsis in the animals. Sham-operated rats were treated identically, except that the cecum was neither ligated nor punctured.

The rats then received either standard therapy (normal saline (30 ml/kg body weight) given once immediately after the CLP procedure to compensate the third-space fluid loss and imipenem (25 mg/kg body weight) daily subcutaneously) or therapy according to the present invention (calcitriol at 25 ng, 100 ng or 400 ng in peanut oil subcutaneously daily). The rats were monitored daily for body weight and survival.

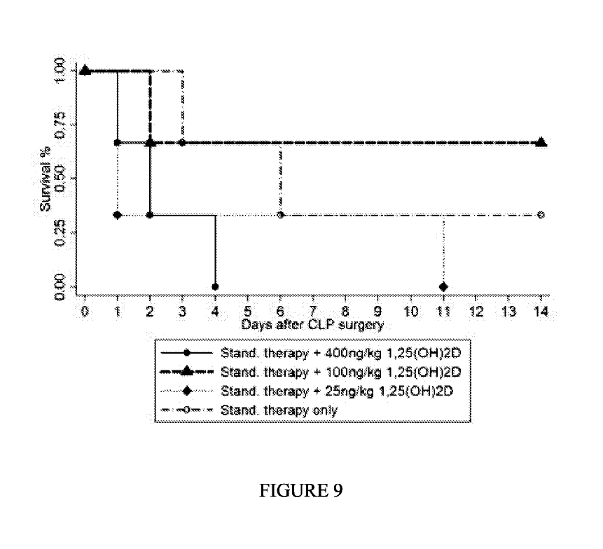

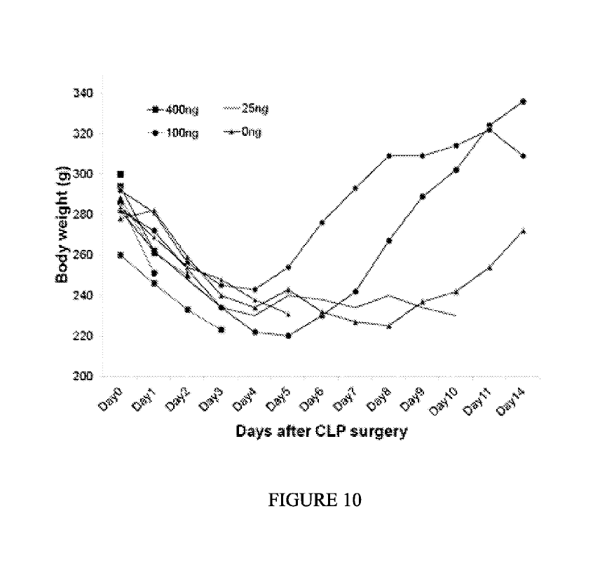

Referring now to FIG. 9 and FIG. 10, there are shown, respectively, a graph of percent survival as a function of time after induction of sepsis in rats showing the effect of various doses of calcitriol (none, triangles; 25 ng daily, line; 100 ng daily, circles; and 400 ng daily, squares) (FIG. 9); and a graph of body weight in grams as a function of time after induction of sepsis in rats showing the effect of various doses of calcitriol (none, triangles; 25 ng daily, line; 100 ng daily, circles; and 400 ng daily, squares) (FIG. 10). As can be seen, calcitriol given at 100 ng daily was the optimal dose for both survival and body weight. When the dose of calcitriol was increased to 400 ng daily, the rats showed typical signs of hypercalcemia and both survival and body weight were negatively affected.

These experiments demonstrate that the optimal dosing for treatment according to the present invention is by an agent, substance or composition that increases vitamin D status but does not cause hypercalcemia.

Although the present invention has been discussed in considerable detail with reference to certain preferred embodiments, other embodiments are possible. Therefore, the scope of the appended claims should not be limited to the description of preferred embodiments contained in this disclosure. All references cited herein are incorporated by reference in their entirety.

1. A method for treating a patient with an episode of sepsis, where treating comprises either decreasing the severity of an episode of sepsis in the patient, or decreasing the likelihood that the patient with sepsis will progress to sepsis with multiple organ dysfunction syndrome, or decreasing the likelihood that the patient with an episode of sepsis will die from the episode of sepsis, or a combination of the preceding, the method comprising:
a) confirming that the patient has an episode of sepsis;
b) determining a blood level of calcium, calcidiol or calcitriol in the patient;
c) determining whether the patient has hypocalcemia, normocalcemia, or hypercalcemia; and
d) administering to the patient, if the patient has hypocalcemia or normocalcemia, one or more than one dose of a composition comprising an agent, wherein the agent is selected from a chemical that raises blood calcium, calcidiol, or calcitriol level in the patient, thereby treating the patient.

2. The method of claim 1, where hypocalcemia is defined as less than 9.0 mg/dL, normocalcemia is defined as between 9.0 and 10.5 mg/dL, and hypercalcemia is defined as greater than 10.5 mg/dL.

3. The method of claim 1, further comprising:
e) determining one or more than one subsequent blood calcium level in the patient; and
f) additionally administering to the patient one or more than one dose of the composition if after the one or more than one subsequent blood calcium level determinations, the patient remains hypocalcemia or normocalcemia, but ceasing additional administration of the composition if after the one or more than one subsequent blood calcium level determinations, the patient becomes hypercalcemia.

4.-9. (canceled)

10. The method of claim 1, where a sub-normal blood calcidiollevel is defined as less than 30 ng/ml, a normal blood calcidiollevel is defined as between 30 and 80 ng/ml, and an elevated blood calcidiol level is defined as greater than 80 ng/ml.

11. (canceled)

12. The method of claim 1, where a sub-normal blood calcitriol level is defined as less than 20 pg/ml, a normal blood calcitriol level is defined as between 20 and 50 pg/ml, and an elevated blood calcitriol level is defined as greater than 50 ng/ml.

13. The method of claim 1, where the one or more than one dose administered is between 0.01 ng and 1 gram, between 0.01 ng and 10,000 meg, between 1 ng and 10,000 mcg, between 0.1 meg and 10,000 meg, between 1 meg and 1,000 meg, between 100 units to 10,000,000 units, between 400 units to 10,000,000 units, between 1000 units to 10,000,000 units, between 1000 units to 100,000 units, between 1000 units to 1,000,000 units, or between 1000 units to 10,000 units.

14.-23. (canceled)

24. The method of claim 1, where the one or more than one dose is administered by a route selected from the group consisting of intrarectal, intramuscular, intraperitoneal, intrathecal, intravenous and oral.

25. The method of claim 1, where the one or more than one dose is a plurality of doses.

26. The method of claim 25, where the plurality of doses is two, three, four, or more than four doses.

27.-30. (canceled)

31. The method of claim 25, where the plurality of doses are administered between one hour and seventy-two hours apart, between one hour and thirty-six hours apart, or between one hour and twenty-four hours apart.

32.-33. (canceled)

34. The method of claim 25, where the plurality of doses are administered twenty-four hours apart.

35. The method of claim 25, where the plurality of doses are 2 meg of paricalcitol administered once daily for twenty-eight days.

36. The method of claim 1, where the agent is selected from the group consisting of 1 alpha-hydroxyergocalciferol; 1-alpha-hydroxyvitamin D2; 1-alpha-hydroxyvitamin D3; 1-alpha(OH)D2; 1-alpha(OH)D3; 1,24,25-trihydroxyvitamin D3; 1,25(OH)2-16-ene-23yne-D3; 19-nor-1,25-dihydroxyvitamin D2; 19-nor-1,25-(OH)2-vitamin D2; 19-nor-1,25-dihydroxyvitamin D3; 20(17→18)-abeo-1α,25-dihydroxy-22-homo-21-norvitamin D(3); 24,25-dihydroxyvitamin D3; alfacalcidiol; calcidiol; calcium; calcitriol; cholecalciferol; doxercalciferol; ergocalciferol; EBI089; HM; KHI060; MC1288 and parathyroid hormone.

37. The method of claim 1, where the agent is a functional analog of one or more than one of the group of 1 alpha-hydroxyergocalciferol; 1-alpha-hydroxyvitamin D2; 1-alpha-hydroxyvitamin D3; 1-alpha(OH)D2; 1-alpha(OH)D3; 1,24,25-trihydroxyvitamin D3; 1,25(OH)2-16-ene-23yne-D3; 19-nor-1,25-dihydroxyvitamin D2; 19-nor-1,25-(OH)2-vitamin D2; 19-nor-1,25-dihydroxyvitamin D3; 20(17→18)-abeo-1α,25-dihydroxy-22-homo-21-norvitamin D(3); 24,25-dihydroxyvitamin D3; alfacalcidiol; calcidiol; calcium; calcitriol; cholecalciferol; doxercalciferol; ergocalciferol; EB1089; HM; KH1060; MC1288 and parathyroid hormone.

38. The method of claim 1, where the agent is calcitriol.

39. The method of claim 1, where the composition comprises at least two agents.

40. (canceled)

41. A method for characterizing the severity of an episode of sepsis in a patient, by determining a likelihood that a patient with an episode of sepsis will progress to sepsis with multiple organ dysfunction syndrome, or by determining a likelihood that a patient with an episode of sepsis will die from the episode of sepsis, the method comprising:
a) confirming that the patient has an episode of sepsis; and
b) determining a blood level of calcium, calcidiol, or calcitriol in the patient;
where a blood level of calcium, calcidiol, or calcitriol below a predetermined amount indicates an increased likelihood that a patient with an episode of sepsis will progress to sepsis with multiple organ dysfunction syndrome, or that the patient will die from the episode of sepsis.

42. The method of claim 41, where the predetermined blood level of calcium is less than 9.0 mg/dL.

43. (canceled)

44. The method of claim 41, where the predetermined blood level of calcidiol is less than 30 ng/ml.

46. The method of claim 41, where the predetermined blood level of calcitriol is less 30 pg/mL, less than 25 pg/mL, less than 20 pg/mL, less than 15 pg/mL, less than 14 pg/mL, less than 13.6 pg/mL, less than 10 pg/mL, or less than 5 pg/mL.

47.-53. (canceled)

54. The method of claim 41, where confirming the patient has an episode of sepsis comprises referring to medical records relevant to the patient.

55. The method of claim 41, where confirming the patient has an episode of sepsis comprises diagnosing the episode of sepsis in the patient.

56. The method of claim 55, where diagnosing the episode of sepsis in the patient comprises determining that the patient has the following criteria: 1) either i) a suspected or confirmed source of infection determined by the treating clinician, or ii) a serum lactate level greater than 2.5 mmol/L or both; and 2) two or more criteria selected from the group consisting of: a) a body temperature greater than 38° C. or less than 36° C.; b) i) a respiratory rate greater than 20 breaths per minute, or ii) partial pressure of carbon dioxide less than 32 mm Hg; c) a heart rate greater than 90 beats per minute; and d) i) a white blood cell count greater than 12,000 cells per mm3, or ii) a white blood cell count less than 4,000 cells per mm3, or iii) a white blood cell count comprising 10% or more than 10% immature forms.

57. The method of claim 56, where determining that the patient has the following criteria comprises measuring the blood level in the patient a plurality of times.

58. The method of claim 57, where the plurality of times is two, three, four, or more than four times.

59.-61. (canceled)

62. The method of claim 41, where the blood level in the patient is measured upon admission to a health care facility.

63. The method of claim 41, where the blood level is measured upon diagnosing the patient with the episode of sepsis.

64. The method of claim 41, where the calcitriol blood level is measured at one or more than one time selected from the group consisting of 0, 24, 48, 72 and 96 hours after admission to a health care facility.

65. The method of claim 41, where the blood level is measured at one or more than one time selected from the group consisting of 0, 24, 48, 72 and 96 hours after diagnosing the patient with the episode of sepsis.

66. The method of claim 41, where the method further comprises determining a presence of one or more than one patient criterion in the patient selected from the group consisting of age, Acute Physiology and Chronic Health Evaluation (APACHE) II scores, and a presence of stroke comorbidity, and where the presence of one or more than one criterion of an age greater than 70 years, an Acute Physiology and Chronic Health Evaluation II score greater than 25, and stroke comorbidity further increases the likelihood that a patient with an episode of sepsis will progress to sepsis with multiple organ dysfunction syndrome, or that the patient will die from the episode of sepsis.
